SEQUENCE LISTING (32 sequences)

SEQ ID NO: 1 - DNA, 27 bp, Artificial Sequence
  Synthetic oligonucleotide primer
cctgtcaacataattggaagaaatmtg

SEQ ID NO: 2 - DNA, 25 bp, Artificial Sequence
  Synthetic oligonucleotide primer
tttaaagtgcaaccaatctgagtca

SEQ ID NO: 3 - DNA, 25 bp, Artificial Sequence
  Synthetic oligonucleotide primer
ccgctgaagaattggaagaaatmtg

SEQ ID NO: 4 - DNA, 29 bp, Artificial Sequence
  Synthetic oligonucleotide primer
ccgctgaagagtgcaaccaatctgagtca

SEQ ID NO: 5 - DNA, 7 bp, Homo sapiens
ttgactc

SEQ ID NO: 6 - DNA, 7 bp, Homo sapiens
atgactc

SEQ ID NO: 7 - DNA, 7 bp, Homo sapiens
gtcaaca

SEQ ID NO: 8 - DNA, 7 bp, Homo sapiens
gtcatca

SEQ ID NO: 9 - DNA, 21 bp, Artificial Sequence
  Synthetic oligonucleotide primer
tcccgcagggttaaaaaagaa

SEQ ID NO: 10 - DNA, 26 bp, Artificial Sequence
  Synthetic oligonucleotide primer
cccacatccagtactgttactgattt

SEQ ID NO: 11 - DNA, 25 bp, Artificial Sequence
  Synthetic oligonucleotide primer
ccgctgaagcagggttaaaaaagaa

SEQ ID NO: 12 - DNA, 28 bp, Artificial Sequence
  Synthetic oligonucleotide primer
ccgctgaagccagtactgttactgattt

SEQ ID NO: 13 - DNA, 6 bp, Homo sapiens
aaaatc

SEQ ID NO: 14 - DNA, 6 bp, Homo sapiens
caaatc

SEQ ID NO: 15 - DNA, 6 bp, Homo sapiens
tttttt

SEQ ID NO: 16 - DNA, 6 bp, Homo sapiens
tttgtt

SEQ ID NO: 17 - DNA, 31 bp, Artificial Sequence
  Synthetic oligonucleotide primer
acaaaatccagacatagttatctatcaatac

SEQ ID NO: 18 - DNA, 27 bp, Artificial Sequence
  Synthetic oligonucleotide primer
agtcagatcctacatacaaatcatcca

SEQ ID NO: 19 - DNA, 25 bp, Artificial Sequence
  Synthetic oligonucleotide primer
ccgctgaagagttatctatcaatac

SEQ ID NO: 20 - DNA, 27 bp, Artificial Sequence
  Synthetic oligonucleotide primer
ccgctgaagctacatacaaatcatcca

SEQ ID NO: 21 - DNA, 5 bp, Homo sapiens
atgga

SEQ ID NO: 22 - DNA, 5 bp, Homo sapiens
gtgga

SEQ ID NO: 23 - DNA, 5 bp, Homo sapiens
catgt

SEQ ID NO: 24 - DNA, 5 bp, Homo sapiens
cacgt

SEQ ID NO: 25 - DNA, 22 bp, Artificial Sequence
  Synthetic oligonucleotide primer
acatctgttgaggtggggattt

SEQ ID NO: 26 - DNA, 25 bp, Artificial Sequence
  Synthetic oligonucleotide primer
tttctgatgttttttgtctggtgtr

SEQ ID NO: 27 - DNA, 25 bp, Artificial Sequence
  Synthetic oligonucleotide primer
ccgctgaaggttgaggtggggattt

SEQ ID NO: 28 - DNA, 25 bp, Artificial Sequence
  Synthetic oligonucleotide primer
ccgctgaagttttttgtctggtgtr

SEQ ID NO: 29 - DNA, 4 bp, Homo sapiens
acca

SEQ ID NO: 30 - DNA, 4 bp, Homo sapiens
taca

SEQ ID NO: 31 - DNA, 4 bp, Homo sapiens
gtaa

SEQ ID NO: 32 - DNA, 4 bp, Homo sapiens
taaa

(19) United States
(12) Patent Application Publication
(10) Pub. No.: US 2009/0286241 A1
(43) Pub. Date: Nov. 19, 2009

(54) SYSTEM AND METHOD FOR DETECTING A GENE MUTATION

(75) Inventors: Shingo Kato, Tokyo (JP); Koji Sudo, Sagamihara-shi (JP)

Correspondence Address: SUGHRUE MION, PLLC, 2100 PENNSYLVANIA AVENUE, N.W., SUITE 800, WASHINGTON, DC, 20037, US

(73) Assignee: SHIMADZU CORPORATION, Kyoto-shi (JP)

(21) Appl. No.: 12/274,029

(22) Filed: Nov. 19, 2008

(30) Foreign Application Priority Data
May 19, 2008 (JP) 2008-131243

(51) Int. Cl. C12Q 1/68; C12M 1/00

(52) U.S. Cl. 435/6; 435/287.2

(57) ABSTRACT

A system for detecting a gene mutation encompasses a spectrum generation mechanism configured to acquire an amplified product containing the specific site sandwiched by recognition sites of a restriction enzyme by using a recognition site introduction-oriented primer, and to generate a mass spectrum of an oligonucleotide fragment, which is cut out from the amplified product by using the restriction enzyme; an area ratio calculation mechanism configured to calculate an area ratio of a peak of a wild-type sequence of the specific site and a peak of a mutation-type sequence of the specific site in the mass spectrum; and an abundance ratio calculation mechanism configured to obtain an abundance ratio of the wild-type sequence and the mutation-type sequence based on a relationship between a previously acquired area ratio and the abundance ratio of the wild-type sequence and the mutation-type sequence.

### CROSS REFERENCE TO RELATED APPLICATION AND INCORPORATION BY REFERENCE

This application claims benefit of priority under 35 USC 119 based on Japanese Patent Application No. P2008-131243, filed on May 19, 2008, the entire contents of which are incorporated by reference herein.

### BACKGROUND OF THE INVENTION

1. Field of the Invention

The present invention relates to an examination technology for a gene mutation, and particularly relates to a system for detecting a gene mutation and a method for detecting a gene mutation.

2. Description of the Related Art

In the anti-HIV therapy, drug resistance of the HIV virus has become a serious problem. In general, an examination of drug-resistant virus has been performed by a direct sequencing method for a resistance-related gene. However, it has been heretofore impossible to detect the resistance-related gene unless such resistant virus is not present in a ratio of 1/3 or more in all viruses. As opposed to this, if it is made possible to detect and quantify drug-resistant virus in a minority group, then it is expected to become possible to acquire important information in grasping a transmission state of the drug-resistant HIV-1 and in selecting a curative medicine in the salvage therapy. In this connection, Hance et.al succeeded in measuring the resistant virus present in a ratio of 0.1% in all the viruses by using an allele-specific real-time PCR (see, for example, J. Virol. 2001 75:6410-6417.). Thereafter, the technology of Hance et.al has been used for researching quantitative changes of the resistant virus in untreated periods in a variety of time periods, maternal infections, strategic treatment interruptions and the like. However, such a method of Hance et.al has had problems that it cannot measure three types of point mutations simultaneously, and that it cannot measure a consecutive two-base mutation (for example, T215Y). Therefore, it has been desired that a novel system for detecting a gene mutation be established.

### SUMMARY OF THE INVENTION

It is an object of the present invention to provide a system for detecting a gene mutation and a method for detecting a gene mutation, which are capable of detecting the gene mutation, and particularly, the gene mutation in the minority group.

An aspect of the present invention inheres in a system for detecting a gene mutation, encompassing (a) a spectrum generation mechanism configured to acquire a first amplified product containing a specific site of a nucleotide sequence so that the specific site is amplified by a first polymerase chain reaction by using a specific site-oriented primer, and a second amplified product containing the specific site sandwiched by recognition sites of a restriction enzyme so that the first amplified product is amplified by a second polymerase chain reaction by using a recognition site introduction-oriented primer containing the recognition sites of the restriction enzyme, and to generate a mass spectrum of an oligonucleotide fragment containing the specific site, the oligonucleotide fragment being cut out from the second amplified product by using the restriction enzyme, (b) an area ratio calculation mechanism configured to calculate a calculated value of an area ratio of a peak of a wild-type sequence of the specific site and a peak of a mutation-type sequence of the specific site in the mass spectrum, (c) a relation storage device configured to store a relationship between a previously acquired area ratio and an abundance ratio of the wild-type sequence and the mutation-type sequence, and (d) an abundance ratio calculation mechanism configured to obtain the abundance ratio of the wild-type sequence and the mutation-type sequence from the calculated value of the area ratio based on the relationship.

Another aspect of the present invention inheres in a method for detecting a gene mutation encompassing (a) acquiring a first amplified product containing a specific site of a nucleotide sequence so that the specific site is amplified by a first polymerase chain reaction by using a specific site-oriented primer, (b) acquiring a second amplified product containing the specific site sandwiched by recognition sites of a restriction enzyme so that the first amplified product is amplified by a second polymerase chain reaction by using a recognition site introduction-oriented primer containing the recognition sites of the restriction enzyme, (c) cutting out an oligonucleotide fragment containing the specific site from the second amplified product by using the restriction enzyme, (d) obtaining a mass spectrum of the cut-out oligonucleotide fragment by a mass spectrometry method, (e) calculating a calculated value of an area ratio of a peak of a wild-type sequence of the specific site and a peak of a mutation-type sequence of the specific site in the mass spectrum, and (f) obtaining an abundance ratio of the wild-type sequence and the mutation-type sequence from the calculated value of the area ratio based on a relationship between a previously acquired area ratio and the abundance ratio of the wild-type sequence and the mutation-type sequence.

### BRIEF DESCRIPTION OF THE DRAWINGS

FIG. 1 is a schematic diagram of a system for detecting a gene mutation according to an embodiment of the present invention;

FIG. 2 is a flowchart showing a method for detecting a gene mutation according to the embodiment of the present invention;

FIG. 3 is a table showing sequences of primers according to an example of the embodiment of the present invention;

FIG. 4 is a mass spectrum according to the example of the embodiment of the present invention;

FIG. 5 is a first chromatogram at 779.5 amu according to the example of the embodiment of the present invention;

FIG. 6 is a second chromatogram at 779,5 amu according to the example of the embodiment of the present invention;

FIG. 7 is a third chromatogram at 779.5 amu according to the example of the embodiment of the present invention;

FIG. 8 is a first chromatogram at 772.0 amu according to the example of the embodiment of the present invention;

FIG. 9 is a second chromatogram at 772.0 amu according to the example of the embodiment of the present invention;

FIG. 10 is a third chromatogram at 772.0 amu according to the example of the embodiment of the present invention;

FIG. 11 is a first graph showing a relationship between a ratio of a drug-resistant mutant strain K2901 and relative detected intensities of oligonucleotide fragments derived from the drug-resistant mutant strain K2901 according to the example of the embodiment of the present invention;

FIG. 12 is a second graph showing the relationship between the ratio of the drug-resistant mutant strain K2901 and the relative detected intensities of the oligonucleotide fragments derived from the drug-resistant mutant strain K2901 according to the example of the embodiment of the present invention;

FIG. 13 is a third graph showing the relationship between the ratio of the drug-resistant mutant strain K2901 and the relative detected intensities of the oligonucleotide fragments derived from the drug-resistant mutant strain K2901 according to the example of the embodiment of the present invention; and

FIG. 14 is a fourth graph showing the relationship between the ratio of the drug-resistant mutant strain K2901 and the relative detected intensities of the oligonucleotide fragments derived from the drug-resistant mutant strain K2901 according to the example of the embodiment of the present invention.

### DETAILED DESCRIPTION OF THE INVENTION

A description will be made below of an embodiment of the present invention. In the following description made with reference to the drawings, the same or similar portions are denoted by the same or similar reference numerals. Note that the drawings are schematic. Hence, specific dimensions and the like should be determined with reference to the following description. Moreover, it is a matter of course that portions different in dimensional relationship and ratio from one another are also included in the drawings.

As shown in FIG. 1, a system for detecting a gene mutation according to the embodiment encompasses a polymerase chain reaction (PCR) device 151, a solid-phase column 153, and a mass spectrometer 152. The PCR device 151 is used for acquiring a first amplified product containing a specific site of a nucleotide sequence so that the specific site of the nucleotide sequence is amplified by a first polymerase chain reaction by using a specific site-oriented primer. Note that, here, the “specific site” is a site in a nucleotide sequence in which a gene mutation as an examination subject may occur. The “specific site-oriented primer” is a PCR-oriented primer for amplifying, by the PCR, the site in the nucleotide sequence in which the gene mutation may occur.

The PCR device 151 is used for acquiring a second amplified product containing the specific site sandwiched by recognition sites of a type-I restriction enzyme so that the first amplified product is amplified by a second polymerase chain reaction by using a recognition site introduction-oriented primer containing the recognition sites of the type-I restriction enzyme. The solid-phase column 153 is used for purifying the second amplified product. The mass spectrometer 152 is used for analyzing an oligonucleotide fragment containing the specific site, which is cut out from the second amplified product by using the type-I restriction enzyme. As the mass spectrometer 152, a liquid chromatograph and the like are usable.

A system for detecting a gene mutation according to the embodiment includes a central processing unit (CPU) 300. The CPU 300 encompasses a spectrum generation mechanism 301 configured to generate a mass spectrum of the oligonucleotide fragment analyzed by the mass spectrometer 152, and an area ratio calculation mechanism 302 configured to calculate a calculated value of an area ratio of a peak of a wild-type sequence of the specific site and a peak of a mutation-type sequence of the specific site in the mass spectrum. To the CPU 300, a relationship storage device 201 is connected, which stores a previously acquired relationship between an area ratio and abundance ratio of the wild-type sequence and the mutation-type sequence. Moreover, the CPU 300 further includes an abundance ratio calculation mechanism 303 configured to obtain a measurement value of the abundance ratio of the wild-type sequence and the mutation-type sequence from the calculated value of the area ratio based on the relationship stored in the relationship storage device 201.

To the CPU 300, there are further connected an input device 312, an output device 313, a program storage device 330, and a temporary storage device 331. As the input device 312, for example, a keyboard, a pointing device such as a mouse, and the like are usable. As the output device 313, an image display device such as a liquid crystal display and a monitor, a printer, and the like are usable. The program storage device 330 stores an operating system for controlling the CPU 300, and the like. The temporary storage device 331 sequentially stores results of arithmetic operations by the CPU 300. As each of the program storage device 330 and the temporary storage device 331, for example, a recording medium for recording the program, such as a semiconductor memory, a magnetic disk, an optical disk, a magneto-optical disk and a magnetic tape, is usable.

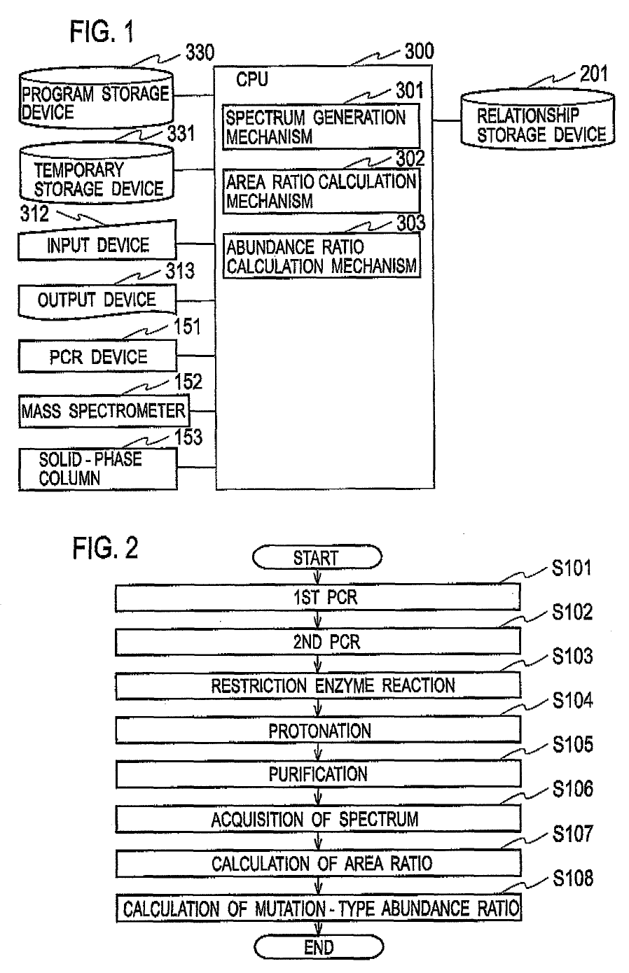

Next, a description will be made of a method for detecting a gene mutation according to the embodiment by using a flowchart shown in FIG. 2. Note that the results of the arithmetic operations by the CPU 300, which are shown in FIG. 1, are sequentially stored in the temporary storage device 331.

(a) In Step S101, a sample containing a cDNA derived from a wild strain and a cDNA derived from a mutant strain is prepared. In comparison with the cDNA derived from the wild strain, a point mutation occurs in the cDNA derived from the mutant strain. Note that a ratio of the cDNA derived from the wild strain and the cDNA derived from the mutant strain in the sample is unknown. Next, a specific site in which a point mutation of the cDNA derived from the wild strain can occur and a specific site in which the point mutation of the cDNA derived from the mutant strain has occurred are amplified by using the specific site-oriented primer and the PCR device 151 shown in FIG. 1, whereby the first amplified product is obtained.

(b) In Step S102, the first amplified product is amplified by using the recognition site introduction-oriented primer containing the recognition sites of the type-I restriction enzyme and the PCR device 151, whereby the second amplified product containing the specific site sandwiched by the recognition sites of the type-I restriction enzyme is obtained. Next, in Step S103, the oligonucleotide fragment containing the specific site is cut out from the second amplified product by using the type-I restriction enzyme. Thereafter, in Step S104, the oligonucleotide fragment is dissolved by ammonium acetate, and in Step S105, the oligonucleotide fragment is purified by the solid-phase column 153. Note that, by dissolving the oligonucleotide fragment by the ammonium acetate, the oligonucleotide fragment becomes likely to be protonated in the subsequent mass spectrometry using electrospray ionization (ESI).

(c) In Step S106, the oligonucleotide fragment is subjected to the mass spectrometry by the mass spectrometer 152. Note that it is possible to perform the mass spectrometry for a plurality of the oligonucleotide fragments at a time. A result of the mass spectrometry is transmitted to the spectrum generation mechanism 301. The spectrum generation mechanism 301 generates the mass spectrometry based on the result of the mass spectrometry. The spectrum generation mechanism 301 transmits the generated mass spectrum to the area ratio calculation mechanism 302,

(d) In Step S107, the area ratio calculation mechanism 302 detects, in the mass spectrum, the peak corresponding to the wild-type 35 sequence of the specific site, and the peak corresponding to the mutation-type sequence of the specific site in which the point mutation has occurred. Next, the area ratio calculation mechanism 802 calculates a peak area of the peak corresponding to the wild-type sequence, and a peak area of the peak corresponding to the mutation-type sequence, and calculates the calculated value of the area ratio of the peak area of the mutation-type sequence with respect to the peak area of the wild-type sequence. The area ratio calculation mechanism 302 transmits the calculated area ratio to the abundance ratio calculation mechanism 303.

(e) In Step S108, the abundance ratio calculation mechanism 303 reads out a linear function indicating a previously acquired relationship between the area ratio of the peak area of the mutation-type sequence with respect to the peak area of the wild-type sequence and the abundance ratio of the mutation-type sequence with respect to the wild-type sequence in the sample. Here, the previously acquired relationship is stored in the relationship storage device 201. Next, the abundance ratio calculation mechanism 303 assigns the calculated value of the area ratio to the linear function, calculates a measurement value of the abundance ratio of the wild-type sequence and the mutation-type sequence, outputs the measurement value to the output device 313, and then ends the method for detecting a gene mutation according to the embodiment.

In accordance with the system for detecting a gene mutation and the method for detecting a gene mutation according to the embodiment described above, it becomes possible to measure the abundance ratio of a trace quantity of the mutation-type sequence contained in the sample.

### EXAMPLES

First, a wild strain IIIB and a drug-resistant mutant strain K2901 separated from blood collected, with the consent, from one of HIV-1 carriers who visit the Keio University Hospital were prepared. The wild strain IIIB does not have a resistance mutation to a reverse transcriptase inhibitor or a protease inhibitor. As opposed to this, the drug-resistant mutant strain K2901 has mutations of M41L, K103N, M184V and T215Y in a reverse transeriptase gene. For example, owing to the mutation of K103N, drug resistance to Lamivudine as a reverse transcriptase inhibitor occurs. Moreover, owing to the mutation of M184V, drug resistance to Efavirenz as a reverse transcriptase inhibitor occurs. Furthermore, owing to the mutation of T215Y, drug resistance to Zidovudine as a reverse transcriptase inhibitor occurs. Moreover, the drug-resistant mutant strain K2901 has mutations of 154V, G73S, V82A and L90M in a protease gene. For example, owing to the mutation of L90M, drug resistance to most of the protease inhibitors occurs.

Next, 60 μl of RNA extract was extracted from 50 pg (containing viruses of approximately 25,000 copies), in conversion to p24 antigen level, of a culture supernatant of each of the wild strain IIIB and the drug-resistant mutant strain K2901 by using QIAamp UltraSens (registered trademark) Virus Kit (made by QIAGEN GmbH). Moreover, to 6 μl of the RNA extract, there were added 2 μl of a random hexamer with 50 μ mmol/l, and 1 μl of dNTPs with 10 mmol/l. Then, an obtained mixture was denatured at 65° C. for 5 minutes, and was thereafter allowed to still stand for 1 minute or more on ice. Furthermore, to the RNA extract, there were added 4 μl of 5×first strand buffer attached to SuperScript (registered trademark) III (made by Invitrogen Corporation), 1 μl of DTT with 0.1 mol/l, 1 μl of SuperScript III with 200 U/ul, and 1 μl of RNasin with 40 U/μl. Then, an obtained mixture was allowed to still stand at room temperature for 5 minutes, at 50° C. for 10 minutes, and at 70° C. for 15 minutes. In such a way cDNA was created.

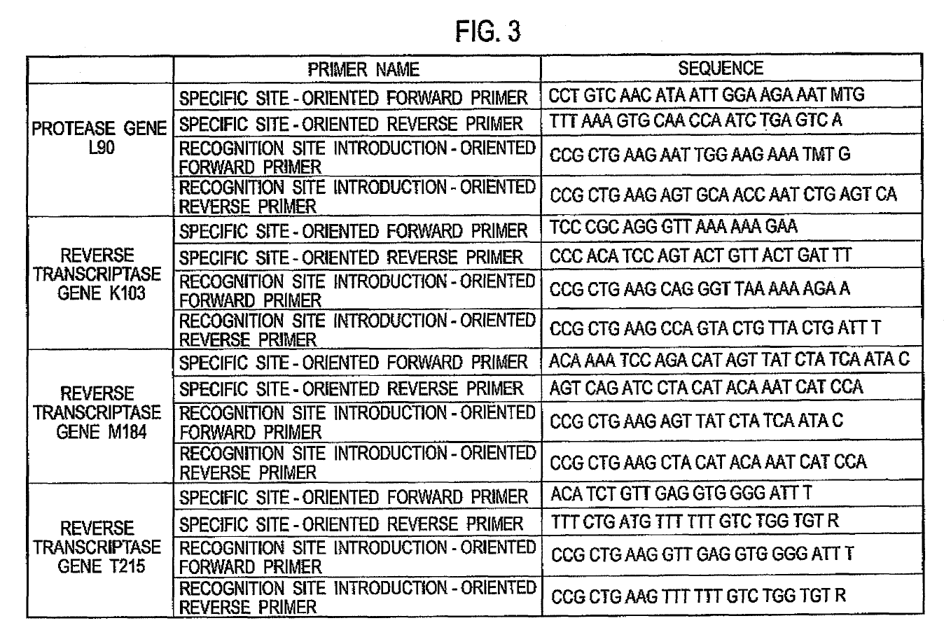

Next, 5 μl of 10×PCR Buffer attached to Platinum (registered trademark) Taq (made by Invitrogen Corporation), 3 μl of MgCl₂ with 50 mmol/l, 0.4 μl of dNTPs with 25 mmol/l, 0.5 μl of a specific site-oriented forward primer with 20 μ mmol/l, 0.5 μl of a specific site-oriented reverse primer with 20 μ mol/l, both of which are shown in FIG. 3, 36.4 μl of distilled water, and 0.2 μl of the Platinum Taq were mixed together, whereby a first PCR-oriented premix was prepared. Thereafter, 46 μl of the first PCR-oriented premix was added to 4 μl of a solution containing the created cDNA, and a resultant was denatured at 94° C. for 2 minutes. Subsequently, for the resultant, a cycle of 94° C. for 5 seconds, 48° C., for 10 seconds, and 72° C. for 15 seconds was repeated 5 times, a cycle of 94° C. for 5 seconds, 60° C. for 10 seconds, and 72° C. for 15 seconds was further repeated 25 times, and finally, a reaction was caused at 72° C. for 1 minute. By the first PCR using the specific site-oriented forward primer and the specific site-oriented reverse primer, a sequence containing a K103 site, a sequence containing a T215 site, and a sequence containing an L90 site are amplified, and the first PCR amplified product is obtained.

Next, 5 μl of the 10×PCR Buffer, 3 μl of MgCl₂ with 50 mmol/l, 0.4 μl of dNTPs with 25 mmol/l, 5 μl of a recognition site introduction-oriented forward primer with 20 μ mol/l, 5 μl of a recognition site introduction-oriented reverse primer with 20 μmol/l, both of which are shown in FIG. 3, 30.1 μl of distilled water, and 0.5 μl of Platinum Taq were mixed together, whereby a second PCR-oriented premix was prepared. Thereafter, 1 μl of the first PCR amplified product was added to 49 μl of the second PCR-oriented premix, and a resultant was denatured at 94° C. for 2 minutes. Subsequently, for the resultant, a cycle of 94° C. for 5 seconds, 60° C. for 10 seconds, and 72° C. for 15 seconds was repeated 30 times, and finally, a reaction was caused at 72° C. for 1 minute. By the second PCR using the recognition site introduction-oriented forward primer and the recognition site introduction-oriented reverse primer, a sequence containing the K103 site sandwiched by recognition sites of a restriction enzyme AcuI, a sequence containing the M184 site sandwiched by the recognition sites of the restriction enzyme AcuI, a sequence containing the T215 site sandwiched by the recognition sites of the restriction enzyme AcuI, and a sequence containing the L90 site sandwiched by the recognition sites of the restriction enzyme AcuI, are amplified, and the second PCR amplified product is obtained.

Next, 5 μl of 10×NE Buffer 2 attached to the restriction enzyme (made by New England Biolabs Japan, Inc.), 2.5 μl of S-adenosyl methionine with 1.6 mmol/l, 7 μl of MgCl₂ with 50 mmol/l, 5 μl of DTT with 10 mmol/l, 28.5 μl of distilled water and 2 μl of the restriction enzyme AcuI were mixed together, whereby a restriction enzyme reaction-oriented premix was prepared. Thereafter, 50 μl of the purified second PCR amplified product was added to 50 μl of the restriction enzyme reaction-oriented premix, and a resultant was allowed to still stand at 37° C. for 1 hour, and at 65° C. for 20 minutes. Then, an oligonucleotide fragment containing the K103 site, an oligonucleotide fragment containing the M184 site, an oligonucleotide fragment containing the T215 site, and an oligonucleotide fragment containing the L90 site, were individually cut out.

Next, 360 μl of 99.5% ethanol was added to 90 μl of a solution containing the cut-out oligonucleotide fragments, and a mixed solution was subjected to centrifugal separation at 14000 rpm for 2 minutes. A supernatant was removed from a resultant, and 10 μl of ammonium acetate with 5 mmol/l was added thereto, whereby the resultant was dissolved. Then, the resultant was purified by using a solid-phase column (ZipTip (registered trademark), made by Nihon Millipore K.K.), and was thereafter dried for 5 minutes in a vacuum dryer. 10 μl of ammonium acetate with 5 mmol/l was added to the dried oligonucleotide fragments, whereby the oligonucleotide fragments were dissolved. Then, a resultant was subjected to the centrifugal separation at 14000 rpm for 2 minutes, whereby 9 μl of a supernatant was collected. The oligonucleotide fragments are protonated by ammonium acetate. 8 μl of the collected supernatant was analyzed by a liquid chromatography-mass spectrometry method (LC-MS). As a liquid chromatograph, HP1100 series HPLC system (made by Agilent Technologies) was used. Moreover, as a mass spectrometer, Q-STAR pulsar i (made by Applied Biosystems) as a quadrupole time-of-flight mass spectrometry (TOF-MS) was used.

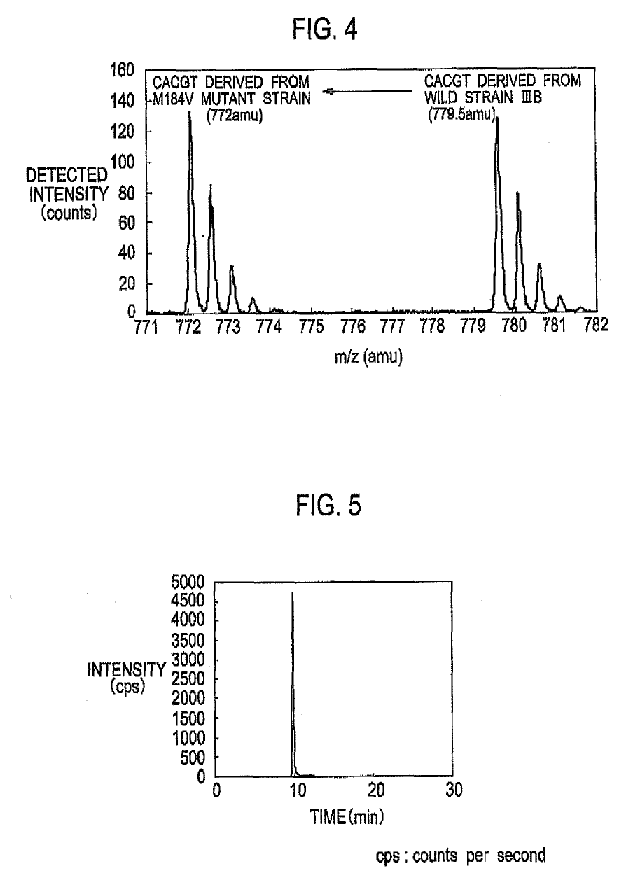

FIG. 4 shows a mass spectrum of the oligonucleotide fragment in the M184 site in the case where the wild strain IIIB and the drug-resistant mutant strain K2901 are mixed together in a ratio of 1:1. Note that m/z in an axis of abscissas represents a ratio of a mass m and a charge z. From the amplified product derived from the wild strain IIIB, an oligonucleotide fragment in which the sequence is CATGT was cut out by the restriction enzyme AcuI, and a peak corresponding to [M-2H]²⁺ was observed at 779.5 amu. As opposed to this, from the amplified product derived from the M184V mutant strain, an oligonucleotide fragment in which the sequence is CACGT was cut out by the restriction enzyme AcuI, and a peak corresponding to [M-2H]²⁺ was observed at 772.0 amu.

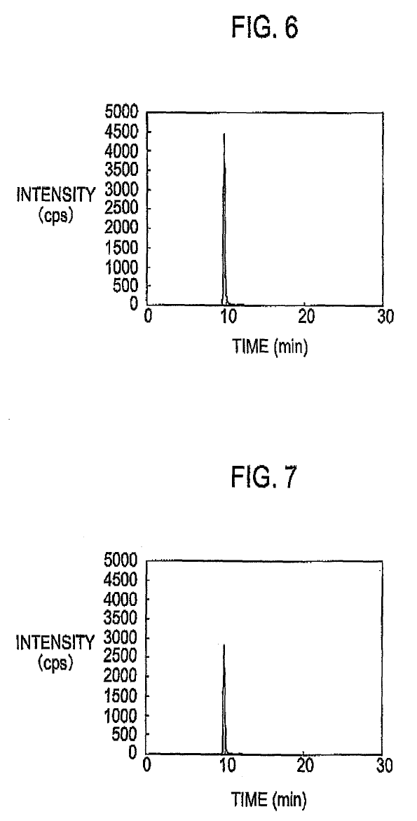

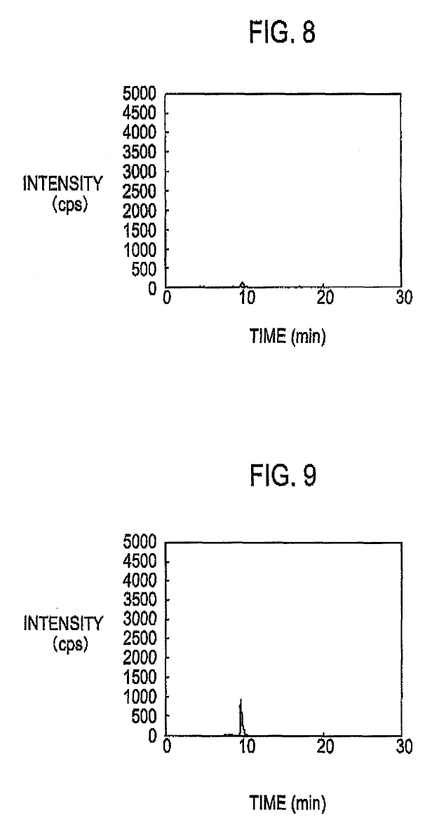

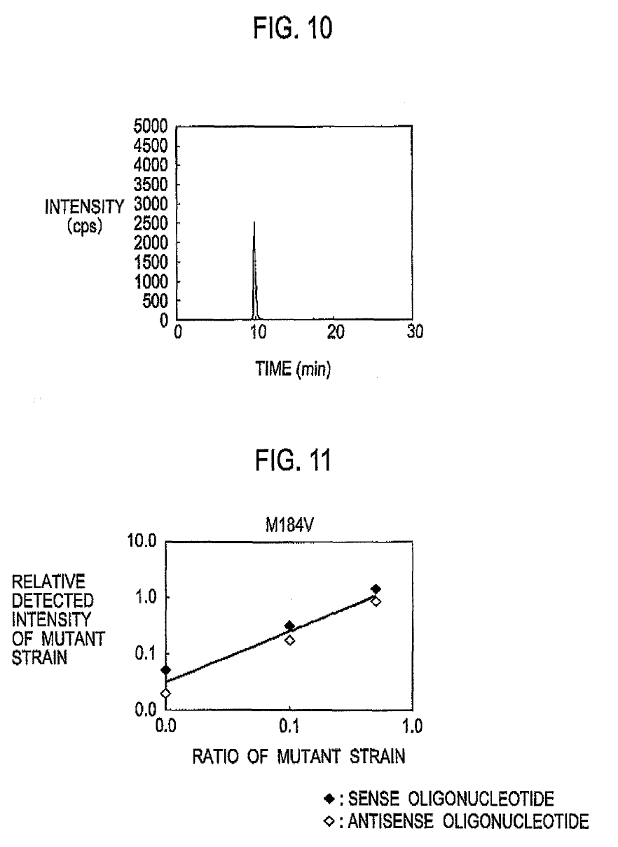

FIG. 5 shows a chromatogram at 779.5 amu in the case where the wild strain IIIB and the drug-resistant mutant strain K2901 are mixed in a ratio of 99:1, FIG. 6 shows a chromatogram at 779.5 amu in the case where the wild strain IIIB and the drug-resistant mutant strain K2901 are mixed in a ratio of 9:1, FIG. 7 shows a chromatogram at 779.5 amu in the case where the wild strain IIIB and the drug-resistant mutant strain K2901 are mixed in a ratio of 1:1, FIG. 8 shows a chromatogram at 772.0 amu in the case where the wild strain IIIB and the drug-resistant mutant strain K2901 are mixed in a ratio of 99:1, FIG. 9 shows a chromatogram at 772.0 amu in the case where the wild strain IIIB and the drug-resistant mutant strain K2901.are mixed in a ratio of 9:1, and FIG. 10 shows a chromatogram at 772,0 amu in the case where the wild strain IIIB and the drug-resistant mutant strain K2901 are mixed in a ratio of 1:1. Heights of the respective peaks at 779.5 amu and 772.0 amu changed in accordance with the mixing ratios of the wild strain IIIB and the drug-resistant mutant strain K2901.

Next, an area surrounded by a plot showing the peak at 779.5 amu and a base line when a detected intensity was 0 in each of FIG. 5 to FIG. 7 was calculated as a wild strain IIIB-specific peak area. Moreover, an area surrounded by a plot showing the peak at 772.0 amu and an axis of abscissas in each of FIG. 8 to FIG. 10 was calculated as a drug-resistant mutant strain K2901-specific peak area. Furthermore, a ratio of the drug-resistant mutant strain K2901-specific peak area with respect to the wild strain IIIB-specific peak area was calculated as a relative detected intensity of the drug-resistant mutant strain K2901. Thereafter, values of the relative detected intensity of the mutant strain in the respective cases where the ratio of the drug-resistant mutant strain K2901 with respect to the wild strain IIIB is 0.01, 0.1 and 1 were graphed as shown in FIG. 11. As shown in FIG. 11, the ratio of the drug-resistant mutant strain K2901 and the relative detected intensity of the drug-resistant mutant strain K2901 had a linear relationship capable of being approximated by a linear function. Hence, it was shown that, if the linear relationship shown in FIG. 11 is previously acquired, then it is possible to calculate the ratio of the drug-resistant mutant strain K2901 from the relative detected intensity of the drug-resistant mutant strain K2901 even if the ratio of the drug-resistant mutant strain K2901 with respect to the wild strain IIIB in the sample is unknown. Moreover, it was shown that it is possible to detect the drug-resistant mutant strain K2901 even if the ratio thereof is 1/100.

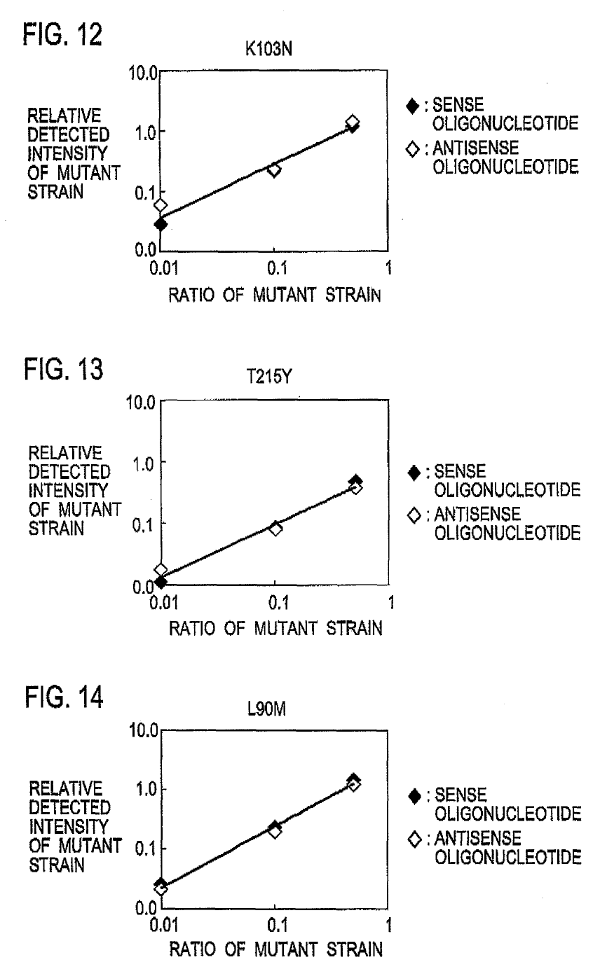

Next, also in the case of calculating a peak area of the chromatogram, which corresponds to the K103N mutation of the reverse transcriptase gene, as the specific peak area of the drug-resistant mutant strain K2901, as shown in FIG. 12, the ratio of the drug-resistant mutant strain K2901 and the relative detected intensity of the drug-resistant mutant strain K2901 had a linear relationship capable of being approximated by a linear function. Moreover, also in the case of calculating a peak area of the chromatogram, which corresponds to the T215Y mutation of the reverse transcriptase gene, as the specific peak area of the drug-resistant mutant strain K2901, as shown in FIG. 13, the ratio of the drug-resistant mutant strain K2901 and the relative detected intensity of the drug-resistant mutant strain K2901 had a linear relationship capable of being approximated by a linear function. Furthermore, also in the case of calculating a peak area of the chromatogram, which corresponds to the L90M mutation of the protease gene, as the specific peak area of the drug-resistant mutant strain K2901, as shown in FIG. 14, the ratio of the drug-resistant mutant strain K2901 and the relative detected intensity of the drug-resistant mutant strain K2901 had a linear relationship capable of being approximated by a linear function.

As described above, the oligonucleotide fragments obtained by cutting the PCR amplified products by the type-I restriction enzyme are analyzed by LC-MS, whereby it becomes possible to detect and quantify the trace quantity of drug-resistant mutant viruses. In particular, it also becomes possible to measure an abundance ratio of the virus having T215Y as an important mutation to the nucleotide reverse transcriptase inhibitor.

### Other Embodiments

The description has been made as above of the present invention based on the embodiment; however, it should not be understood that the description and the drawings, which compose a part of this disclosure, limit this invention. For example, though the oligonucleotide fragment is purified by the solid-phase column in Step S105 of FIG. 2, Step S105 may be omitted. However, in the case of adopting Step S105, it becomes possible to detect the drug-resistant mutant strain in the sample even if the ratio thereof is 1/500. Meanwhile, in the case of omitting Step S105, it becomes possible to detect the drug-resistant mutant strain in the sample up to no more than a ratio of approximately 1/100.

Moreover, in Example, by using the specific site-oriented primer, the specific site of the nucleotide sequence was amplified by the first PCR, whereby the first amplified product containing the specific site was acquired. Thereafter, by using the recognition site introduction-oriented primer, the first amplified product was amplified by the second PCR, whereby the second amplified product containing the specific site sandwiched by the recognition sites of the restriction enzyme was acquired. The reason for the above is as follows. Specifically, a mutated gene derived from an HIV-1 carrier has a plurality of the mutations, and accordingly, if the first PCR is omitted, then there occurs a difference in amplification factor of the PCR between the nucleotide derived from the mutant strain and the nucleotide derived from the wild strain owing to a difference therebetween in homology to the recognition site introduction-oriented primer. However, in the case where there is no difference in homology to the recognition site introduction-oriented primer, the first PCR may be omitted, and the specific site of the nucleotide sequence may be amplified by using the recognition site introduction-oriented primer from the beginning.

Moreover, in Example, the detection example of the mutant strain carried by the HIV-1 carrier has been shown; however, it is a matter of course that the system for detecting a gene mutation and the method for detecting a gene mutation according to the embodiment of the present invention are applicable not only to the analysis of the ratio of the cDNA mutation but also to detection of a ratio of a DNA mutation of virus or bacteria, and further, to detection of duplication, deletion and the like of SNPs and genes of a cell.

From this disclosure as described above, a variety of alternative embodiments, examples and application technologies will be obvious for those skilled in the art. Hence, the technical scope of the present invention is defined only by items which specify the invention, and are according to the scope of claims reasonable from the above description.

### Sequence List

Sequence numbers 1 to 32 described in a sequence table of this specification denote the following sequences.
[Sequence number: 1] Base sequence of specific site-oriented forward primer for amplifying sequence containing L90 site of protease gene
[Sequence number: 2] Base sequence of specific site-oriented reverse primer for amplifying sequence containing L90 site of protease gene
[Sequence number: 3] Base sequence of recognition site introduction-oriented forward primer for introducing recognition site of restriction enzyme AcuI into amplified product of sequence containing L90 site of protease gene
[Sequence number: 4] Base sequence of recognition site introduction-oriented reverse primer for introducing recognition site of restriction enzyme AcuI into amplified product of sequence containing L90 site of protease gene
[Sequence number: 5] Sense sequence of oligonucleotide fragment derived from wild strain, which contains L90 site and is cut out by restriction enzyme AcuI
[Sequence number: 6] Sense sequence of oligonucleotide fragment derived from mutant strain, which contains L90M-mutated site and is cut out by restriction enzyme AcuI
[Sequence number: 7] Anti-sense sequence of oligonucleotide fragment derived from wild strain, which contains L90 site and is cut out by restriction enzyme AcuI
[Sequence number: 8] Anti-sense sequence of oligonucleotide fragment derived from mutant strain, which contains L90M-mutated site and is cut out by restriction enzyme AcuI
[Sequence number: 9] Base sequence of specific site-oriented forward primer for amplifying sequence containing K103 site of reverse transcriptase gene
[Sequence number: 10] Base sequence of specific site-oriented reverse primer for amplifying sequence containing K103 site of reverse transcriptase gene
[Sequence number: 11] Base sequence of recognition site introduction-oriented forward primer for introducing recognition site of restriction enzyme AcuI into amplified product of sequence containing K103 site of reverse transcriptase gene
[Sequence number; 12] Base sequence of recognition site introduction-oriented reverse primer for introducing recognition site of restriction enzyme AcuI into amplified product of sequence containing K103 site of reverse transcriptase gene
[Sequence number: 13] Sense sequence of oligonucleotide fragment derived from wild strain, which contains K103 site and is cut out by restriction enzyme AcuI
[Sequence number: 14] Sense sequence of oligonucleotide fragment derived from mutant strain, which contains K103N-mutated site and is cut out by restriction enzyme AcuI
[Sequence number: 15] Anti-sense sequence of oligonucleotide fragment derived from wild strain, which contains K103 site and is cut out by restriction enzyme AcuI
[Sequence number: 16] Anti-sense sequence of oligonucleotide fragment derived from mutant strains which contains K103N-mutated site and is cut out by restriction enzyme AcuI
[Sequence number: 17] Base sequence of specific site-oriented forward primer for amplifying sequence containing M184 site of reverse transcriptase gene
[Sequence number: 18] Base sequence of specific site-oriented reverse primer for amplifying sequence containing M184 site of reverse transcriptase gene
[Sequence number: 19] Base sequence of recognition site introduction-oriented forward primer for introducing recognition site of restriction enzyme AcuI into amplified product of sequence containing M184 site of reverse transcriptase gene
[Sequence number: 20] Base sequence of recognition site introduction-oriented reverse primer for introducing recognition site of restriction enzyme AcuI into amplified product of sequence containing M184 site of reverse transcriptase gene
[Sequence number: 21] Sense sequence of oligonucleotide fragment derived from wild strain, which contains M184 site and is cut out by restriction enzyme AcuI
[Sequence number: 22] Sense sequence of oligonucleotide fragment derived from mutant strain, which contains M184V-mutated site and is cut out by restriction enzyme AcuI
[Sequence number: 23] Anti-sense sequence of oligonucleotide fragment derived from wild strain, which contains M184 site and is cut out by restriction enzyme AcuI
[Sequence number: 24] Anti-sense sequence of oligonucleotide fragment derived from mutant strain, which contains M184V-mutated site and is cut out by restriction enzyme AcuI
[Sequence number: 25] Base sequence of specific site-oriented forward primer for amplifying sequence containing T215 site of reverse transcriptase gene
[Sequence number: 26] Base sequence of specific site-oriented reverse primer for amplifying sequence containing T215 site of reverse transcriptase gene
[Sequence number: 27] Base sequence of recognition site introduction-oriented forward primer for introducing recognition site of restriction enzyme AcuI into amplified product of sequence containing T215 site of reverse transcriptase gene
[Sequence number: 28] Base sequence of recognition site introduction-oriented reverse primer for introducing recognition site of restriction enzyme AcuI into amplified product of sequence containing T215 site of reverse transcriptase gene
[Sequence number: 29] Sense sequence of oligonucleotide fragment derived from wild strain, which contains T215 site and is cut out by restriction enzyme AcuI
[Sequence number: 30] Sense sequence of oligonucleotide fragment derived from mutant strain, which contains T215Y-mutated site and is cut out by restriction enzyme AcuI
[Sequence number: 31] Anti-sense sequence of oligonucleotide fragment derived from wild strain, which contains T215 site and is cut out by restriction enzyme AcuI
[Sequence number: 32] Anti-sense sequence of oligonucleotide fragment derived from mutant strain, which contains T215Y-mutated site and is cut out by restriction enzyme AcuI

In the case of displaying the bases by abbreviations in this specification, abbreviations by IUPAC-IUB Commission on Biochemical Nomenclature, or idiomatic abbreviations in the field concerned are used. Examples of the abbreviations are shown below. a: adenine, t: thymine, g: guanine, c: cytosine, u: uracil, m: adenine or cytosine, r: guanine or adenine

What is claimed is:

1. A system for detecting a gene mutation comprising:
a spectrum generation mechanism configured to acquire a first amplified product containing a specific site of a nucleotide sequence so that the specific site is amplified by a first polymerase chain reaction by using a specific site-oriented primer, and a second amplified product containing the specific site sandwiched by recognition sites of a restriction enzyme so that the first amplified product is amplified by a second polymerase chain reaction by using a recognition site introduction-oriented primer containing the recognition sites of the restriction enzyme, and to generate a mass spectrum of an oligonucleotide fragment containing the specific site, the oligonucleotide fragment being cut out from the second amplified product by using the restriction enzyme;
an area ratio calculation mechanism configured to calculate a calculated value of an area ratio of a peak of a wild-type sequence of the specific site and a peak of a mutation-type sequence of the specific site in the mass spectrum; and
an abundance ratio calculation mechanism configured to obtain an abundance ratio of the wild-type sequence and the mutation-type sequence from the calculated value of the area ratio based on a relationship between a previously acquired area ratio and the abundance ratio of the wild-type sequence and the mutation-type sequence.

2. A system for detecting a gene mutation comprising:
a spectrum generation mechanism configured to acquire a amplified product containing a specific site sandwiched by recognition sites of a restriction enzyme so that the specific site of a nucleotide sequence is amplified by a polymerase chain reaction by using a recognition site introduction-oriented primer containing the recognition sites of the restriction enzyme, and to generate a mass spectrum of an oligonucleotide fragment containing the specific site, the oligonucleotide fragment being cut out from the amplified product by using the restriction enzyme;
an area ratio calculation mechanism configured to calculate a calculated value of an area ratio of a peak of a wild-type sequence of the specific site and a peak of a mutation-type sequence of the specific site in the mass spectrum; and
an abundance ratio calculation mechanism configured to obtain an abundance ratio of the wild-type sequence and the mutation-type sequence from the calculated value of the area ratio based on a relationship between a previously acquired area ratio and the abundance ratio of the wild-type sequence and the mutation-type sequence.

3. The system of claim 1, wherein the restriction enzyme is a type-I restriction enzyme.

4. The system of claim 1, wherein the spectrum generation mechanism contains a liquid chromatograph-mass spectrometer.

5. The system of claim 1, wherein the cut-out oligonucleotide fragment is dissolved in ammonium acetate.

6. The system of claim 1, further comprising a solid-phase column configured to purify the cut-out oligonucleotide fragment.

7. The system of claim 1, wherein the relationship is indicated by a linear function.

8. A method for detecting a gene mutation comprising:
acquiring a first amplified product containing a specific site of a nucleotide sequence so that the specific site is amplified by a first polymerase chain reaction by using a specific site-oriented primer;
acquiring a second amplified product containing the specific site sandwiched by recognition sites of a restriction enzyme so that the first amplified product is amplified by a second polymerase chain reaction by using a recognition site introduction-oriented primer containing the recognition sites of the restriction enzyme;
cutting out an oligonucleotide fragment containing the specific site from the second amplified product by using the restriction enzyme;
obtaining a mass spectrum of the cut-out oligonucleotide fragment by a mass spectrometry method;
calculating a calculated value of an area ratio of a peak of a wild-type sequence of the specific site and a peak of a mutation-type sequence of the specific site in the mass spectrum; and
obtaining an abundance ratio of the wild-type sequence and the mutation-type sequence from the calculated value of the area ratio based on a relationship between a previously acquired area ratio and the abundance ratio of the wild-type sequence and the mutation-type sequence.

9. A method for detecting a gene mutation comprising:
acquiring a amplified product containing a specific site sandwiched by recognition sites of a restriction enzyme so that the specific site of a nucleotide sequence is amplified by a polymerase chain reaction by using a recognition site introduction-oriented primer containing the recognition sites of the restriction enzyme;
cutting out an oligonucleotide fragment containing the specific site from the amplified product by using the restriction enzyme;
obtaining a mass spectrum of the cut-out oligonucleotide fragment by a mass spectrometry method;
calculating a calculated value of an area ratio of a peak of a wild-type sequence of the specific site and a peak of a mutation-type sequence of the specific site in the mass spectrum; and
obtaining an abundance ratio of the wild-type sequence and the mutation-type sequence from the calculated value of the area ratio based on a relationship between a previously acquired area ratio and the abundance ratio of the wild-type sequence and the mutation-type sequence.

10. The method of claim 8, wherein the restriction enzyme is a type-I restriction enzyme.

11. The method of claim 8, wherein the mass spectrometry method is a liquid chromatograph-mass spectrometry method.

12. The method of claim 8, further comprising:
dissolving the cut-out oligonucleotide fragment with ammonium acetate.

13. The method of claim 8, further comprising
purifying the cut-out oligonucleotide fragment by a solid-phase column.

14. The method of claim 8, wherein the relationship is indicated by a linear function.

15. The method of claim 8, wherein mass spectra of a plurality of the cut-out oligonucleotide fragments are obtained at a time on the step of obtaining the mass spectrum.
